(19) United States
(12) Patent Application Publication
(10) Pub. No.: US 2026/0283885 A1
(43) Pub. Date: Sep. 24, 2026

(54) APPARATUS, SYSTEMS, AND METHODS FOR HYPERBARIC THERMOREGULATED MEDICAL EVACUATION POD

(71) Applicant: TOWSTER CORPORATION, MONTEBELLO, CA (US)

(72) Inventors: Yousef Ahmed, Montebello, CA (US); Andre Savadjian, Whippany, NJ (US)

(73) Assignee: TOWSTER CORPORATION, MONTEBELLO, CA (US)

(21) Appl. No.: 19/471,644

(22) PCT Filed: Nov. 4, 2024
(86) PCT No.: PCT/US2024/054454

§ 371 Date: Oct. 1, 2025

Related U.S. Application Data
(60) Provisional application No. 63/596,014, filed on Nov. 3, 2023.

(51) Int. Cl. A61G 10/02; B63C 11/32

(52) U.S. Cl. CPC A61G 10/026; B63C 11/32; A61G 2220/00

(57) ABSTRACT

In some embodiments, a hyperbaric medical evaluation chamber includes a frame configured to transition between a collapsed configuration to an expanded configuration. The hyperbaric medical evacuation chamber further includes a collapsible shell coupled to the frame, defining an internal compartment when the frame is in the expanded configuration. The hyperbaric medical evacuation pod further includes a collapsible rail disposed inside the collapsible shell and coupled to the frame. The collapsible rail may be configured to accept a backboard. The hyperbaric medical evacuation chamber may further include a lid removably couplable to the collapsible shell at the first end to hermetically seal the collapsible shell when the frame is in the expanded configuration.

### CROSS REFERENCE TO RELATED APPLICATIONS

This application claims priority to and benefit of provisional U.S. Patent Application No. 63/596,014, filed Nov. 3, 2023, the entire disclosure of which is hereby incorporated by reference.

### FIELD

The embodiments described herein relate generally to hyperbaric oxygen therapy and more particularly, to systems, devices, and methods for using hyperbaric thermoregulated medical evacuation pods.

### BACKGROUND

Hyperbaric chambers are specialized medical devices that deliver oxygen at pressures higher than atmospheric levels. Delivering oxygen at an increased pressure can enhance tissue oxygenation and oxygen absorption by the bloodstream. Within a body, increased oxygen promotes cellular repair, accelerates tissue healing, reduces inflammation, and supports immune function. Hyperbaric oxygen therapy (HBOT), where persons are subject to therapy in hyperbaric chambers, has been used to treat conditions such as decompression sickness for divers, carbon monoxide poisoning, air embolisms, and non-healing wounds. In recent trends, HBOT has shown effectiveness for treating other conditions including crush injuries, traumatic brain injuries, severe infections, burns, severe anemia, various shock states, and other ailments. Typical hyperbaric chambers are large, stationary units that use dedicated infrastructure, which limits their availability, particularly in time-sensitive or mobile healthcare scenarios.

In remote areas, medical evacuation (MEDEVAC), and/or casualty evacuation (CASEYAC) operations, the ability to deliver immediate, effective treatment can mean the difference between life and death, especially in the period following injury before and/or during patient transport to an advanced medical facility within the “golden hour”. Patients suffering from hypoxia, decompression sickness, lactic acidosis, various shock states, or another traumatic injury may benefit greatly from HBOT, however, current systems are impractical for field use due to their size, weight, and operational requirements. Typical hyperbaric chambers typically utilize a fixed installation, stable power sources, and a combination of connections to create a highly controlled environment. Typical hyperbaric chambers, due to their stationary nature, are incompatible with unpredictable and fast-paced environments associated with emergency evacuation.

Portability is an important aspect of MEDEVAC and CASEYAC situations, such as missions, disaster response, search-and-rescue, and other emergency medical services operating in remote areas. Portability can be driven by ease of use, durability, compatibility, weight, and other factors. Additionally, the concept of the “golden hour” is an important compounding consideration, where providing medical care within the first hour after an injury increases the chances of saving the life of a patient. Initiating HBOT early, and within the golden hour, could significantly improve outcomes by stabilizing patients before the patient can reach comprehensive medical care.

There are two primary types of hyperbaric chambers: monoplace and multiplace. Monoplace chambers are designed to accommodate a single patient and are usually pressurized with 100% oxygen. Relative to multiplace chambers, monoplace chambers are typically smaller, more compact, and simpler to operate making them suitable for individual therapy. When using monoplace chambers, patients lie inside a tube-like enclosure where they can receive HBOT. Monoplace chambers are typically less resource intensive than their multiplace counterparts, but their limited capacity and size restrict utility and are resistant to adjustments that may need to be made during the HBOT session.

Conversely, multiplace chambers are relatively larger enclosures capable of treating multiple patients at once. These chambers are typically pressurized with air, and patients breathe 100% oxygen through a mask or hood. Multiplace chambers are versatile, as medical personnel can enter the chamber to monitor and assist patients during HBOT. Multiplace chambers are often used in hospitals and specialized care centers to handle complex cases or treat multiple individuals at a time. However, multiplace chambers are large, heavy, require more infrastructure, and are impractical for emergency applications.

Thus, a need exists for hyperbaric oxygen therapy that can function with MEDEVAC use by combining portability while addressing the challenges of field and transport environments.

### SUMMARY

In some embodiments, a hyperbaric medical evaluation chamber includes a frame configured to transition from a collapsed configuration to an expanded configuration. The hyperbaric medical evacuation chamber further includes a collapsible shell coupled to the frame, defining an internal compartment when the frame is in the expanded configuration. The hyperbaric medical evacuation pod further includes a collapsible rail disposed inside the collapsible shell and coupled to the frame. The collapsible rail may be configured to accept a backboard, where a patient may lie on and be inserted and removed from the chamber with ease. The hyperbaric medical evacuation chamber may further include a lid removably couplable to the collapsible shell at end to hermetically seal the collapsible shell when the frame is in the expanded configuration.

### BRIEF DESCRIPTION OF THE DRAWINGS

FIG. IA schematically depicts a hyperbaric medical chamber in a collapsed configuration, according to an embodiment.

FIG. IB schematically depicts a hyperbaric medical evacuation chamber of FIG. IA in an extended configuration.

FIG. 2 schematically depicts a hyperbaric medical chamber in an extended configuration, according to an embodiment.

FIG. 3 depicts an illustration of a hyperbaric medical chamber in a collapsed configuration, according to an embodiment.

FIG. 4 depicts a perspective view of a hyperbaric medical chamber with a user inside, according to an embodiment.

FIG. 5 depicts a bottom view of the hyperbaric medical chamber of FIG. 4, according to another embodiment. of FIG. 4.

FIG. 6 depicts an alternate perspective view of the hyperbaric medical chamber

FIG. 7 depicts a hyperbaric medical chamber in an expanded configuration as well as a collapsed configuration for portability, according to an embodiment.

FIG. 8 depicts a perspective view of a hyperbaric medical chamber in a collapsed and expanded configuration, according to an embodiment.

FIG. 9 depicts a perspective exploded view of a hyperbaric medical chamber, according to an embodiment.

FIG. IO depicts an illustration of a hyperbaric medical chamber configured to operate with a mobilizer, according to an embodiment.

### DETAILED DESCRIPTION

The embodiments described herein relate generally to systems, devices, and/or methods for hyperbaric oxygen therapy. In some embodiments, a hyperbaric medical device may be configured to deliver hyperbaric oxygen therapy in a field-based situation as opposed to a clinical setting. For example, a hyperbaric medical device can include a transportable and collapsible pod configured for the transportation and delivery of hyperbaric oxygen therapy.

The terminology used herein is for the purpose of describing particular embodiments, implementations, and/or concepts (including any feature(s) or aspect(s) thereof) and is not intended to be limiting. Unless defined otherwise, technical and/or scientific terms used herein have the same meanings as commonly understood by one of ordinary skills in the art. Any explanation or discussion of or using particular terms is intended to provide context and to facilitate understanding and is not necessarily intended to replace or supersede commonly used or known definitions understood by one skilled in the art unless explicitly stated otherwise. Moreover, various terms may be used to describe similar or substantially the same embodiments, implementations, and/or concepts (including any feature(s) or aspect(s) thereof) and thus, the use of the particular term is not intended to be limiting and/or to the exclusion of other terms unless the terms are mutually exclusive, or the context clearly states otherwise.

As used herein, the singular forms “a,” “an,” and “the” are intended to include the plural forms as well, unless the context clearly indicates otherwise. With respect to the use of singular and/or plural terms herein, those having skill in the art can translate from the singular to the plurality and/or vice versa as is appropriate for the context and/or application. Furthermore, any reference herein to a singular component, feature, aspect, etc. is not intended to imply the exclusion of more than one such component, feature, aspect, etc. (and/or vice versa) unless expressly stated otherwise. The various singular/plural permutations may be expressly set forth herein for the sake of clarity.

In general, terms used herein and in the appended claims are intended as “open” terms unless expressly stated otherwise. For example, the term “including” should be interpreted as “including but not limited to,” the term “having” should be interpreted as “having at least,” etc. Similarly, the term “comprising” may specify the presence of stated features, elements, components, integers (or fractions thereof), steps, operations, and/or the like but does not preclude the presence or addition of one or more other features, elements, components, integers (or fractions thereof), steps, operations, elements, components, and/or groups thereof, and/or the like unless such combinations are otherwise mutually exclusive.

As used herein the term “and/or” includes any and all combinations of one or more of the associated listed items. It should be understood that any suitable disjunctive word and/or phrase presenting two or more alternative terms, whether in the written description or claims, contemplate the possibilities of including one of the terms, either of the terms, or both/all of the terms. For example, the phrase “A and/or B” will be understood to include the possibilities of “A” alone, “B” alone, or a combination of “A and B.”

All ranges described herein include each individual member or value and are intended to encompass any and all possible subranges and/or combinations of subranges thereof unless expressly stated otherwise. Any listed range should be recognized as sufficiently describing and enabling the same range being broken down into at least equal subparts unless expressly stated otherwise.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

As used herein, the words “proximal” and “distal” refer to the direction closer to and away from, respectively, a user who would place the device into contact with a patient. The words “proximal” or “distal” can be relative terms and do not necessarily refer to universally fixed positions or directions. Thus, for example, the end or end portion of a device first touching the body of the patient would be the distal end or distal end portion, while the opposite end or end portion of the device (e.g., the end or end portion of the device being manipulated by the user) would be the proximal end or proximal end portion of the device.

The embodiments, methods, and/or implementations herein, and/or the various features or advantageous details thereof, are explained more fully with reference to the non-limiting examples illustrated in the accompanying drawings and detailed in the following description. The examples and/or embodiments described herein are intended to facilitate an understanding of structures, functions, and/or aspects of the embodiments, ways in which the embodiments may be practiced, and/or to further enable those skilled in the art to practice the embodiments herein. Similarly, methods and/or ways of using or implementing the embodiments described herein are provided by way of example only and not limitation. Specific uses and/or implementations described herein are not provided to the exclusion of other uses unless the context expressly states otherwise. Descriptions of well-known components, methods, techniques, etc. may be omitted so as to not obscure the embodiments herein. Like numbers refer to like elements throughout.

Turning now to the figures, FIG. IA is a block diagram of an example portable hyperbaric medical chamber 100, also referred to herein as a hyperbaric MEDEVAC chamber and/or a pod, in a collapsed configuration. FIG. IB is a block diagram ofhyperbaric medical chamber 100 in an expanded configuration. hyperbaric medical chamber I00 includes a frame 105, a shell 110, a rail 115, a first lid 120, a second lid 125, and a valve 130. The hyperbaric medical chamber 100 can be portable and/or suitable for use for MEDEVAC. The hyperbaric medical chamber 100 may include other subsystems and/or be configured to be compatible with other subsystems, such as a gas supply system, patient monitoring system, thermoregulation system, medication delivery system, etc.

In operation, the shell 110 serves as a flexible, durable membrane used to contain and/or regulate pressurized fluids while creating a capsule. The shell 110 can be made of lightweight material designed for strength, puncture resistance, and reduced gas permeability. In some embodiments, the shell can be rigid (e.g., telescoping rings), but it may be preferable that the shell be flexible (e.g., fabric-like) and configured to collapse, fold, roll up, and/or deform to move into the collapsed configuration. Similarly stated, in some embodiments, the shell can support loads in tension, but not compression. For example, the shell 110 may be made of Vectran™, high-performance polymer fiber material, manufactured fiber, high-performance multifilament yarn spun from liquid crystal polymer, or a similar material. The lightweight and resilient nature of the shell 110 facilitates the use of the hyperbaric medical chamber 100 in demanding applications. The shell 110 and/or endcaps (discussed below with reference to lids 120, 125) can, in some embodiments, be the principal structural components when the hyperbaric medical chamber 100 is in the expanded configuration and pressurized. Similarly stated, the shell 110 and/or endcaps can be operable to resist hoop and/or radial, and/or axial stresses associated with pressurizing the hyperbaric medical chamber 100 to, for example, 2-4 atm absolute and/or 0.2-5 atm gauge.

In operation, the frame 105 provides structural support and allows the hyperbaric medical chamber 100 to move between the expanded and collapsed configurations. In some embodiments the frame 105 can be constructed of spring steel or any other suitable material. The frame 105 may be collapsible along a length, L, or in diameter, D. Mechanisms to achieve collapsibility may include, but are not limited to a spiral spring-like expandable member, a plurality of interconnected members, telescopic components, or other suitable collapsible/expandable mechanisms. In the collapsed configuration, the frame 105 provides stability, structure, and, in some embodiments, keeps the hyperbaric medical chamber 100 in a substantially cylindrical configuration. In the collapsed configuration and/or in transport, the frame 105 can provide distribution of applied forces such as weight, stress, or environmental loads across the frame to prevent deformation or failure. In some embodiments, in the expanded configuration, the frame 105 provides structural stability and resists outward (e.g., radial and hoop) forces associated with a pressurized interior of the pod 100 in the expanded configuration. Similarly stated, in some embodiments, the frame 105 can reinforce the shell 110, completely or partially define the shape of the pod 100 in the expanded configuration and provide a stronger pressure vessel than the shell 110 alone. The frame 105 can be internal to or external to the shell. The frame 105 can be coupled to the shell via any suitable means. For example, the frame 105 can be attached to the shell 110 via sleeves formed in/defined by the shell, ties, loops, stitching, hook-and-loop fasteners, welds (solvent or metallurgic), rivets, screws, etc.

Rail 115 is a support structure configured to receive a backboard, when the pod 100 is in the expanded configuration, as discussed in further detail herein. For example, the rail 115 can enable the insertion of a patient by having an inner notch to accept a backboard, a patient support board, a stretcher, or other patient carrier. By allowing the patient carrier to slide along the length, L, of the rail 115, the patient may be inserted and/or removed with ease into or out of the hyperbaric medical chamber 100. The rail 115 and/or backboard can further compartmentalize the hyperbaric medical chamber I00, for example defining a top, patient portion of the hyperbaric medical chamber 100 and a bottom, utility portion of the hyperbaric medical chamber 100 (e.g., containing/configured to contain pressurized gas containers and similar components). In addition, the rail can provide stability to the hyperbaric medical chamber 100. Like frame 105, the rail 115 can move between a collapsed configuration and an expanded configuration, for example, through telescopic, segmentation, or other suitable techniques.

The first lid 120 and/or the second lid 125 provide mechanisms by which to hermetically seal the hyperbaric medical chamber 100. In some embodiments, the first lid 120 is removably couplable to the frame 105 (e.g., the first lid 120 can be completely removed from hyperbaric medical chamber I00) via an endcap. The hermetic seal formed between first lid 120 and hyperbaric medical chamber 100, when the hyperbaric medical chamber 100 is in use (e.g., in the expanded configuration), allows the interior of the hyperbaric medical chamber 100 to be pressurized. Endcap(s) (not shown in FIG. 1) can be a metal or other suitable band of material coupled to the shell 110 that acts as a structural member and/or provides a mating surface for the lids 120, 125. Similarly stated, the lid(s) 120, 125 can transfer axial stresses associated with pressurizing the hyperbaric medical chamber 100 to shell 110 and/or frame 105 via the lid(s) 120, 125 and/or vice versa. In some embodiments, the second lid 125 is permanently affixed to the frame I 05 so as to form one contiguous component. In other embodiments, the second lid 125 is removably coupled to the frame, similar to the first lid 120. Conversely, in some embodiments, the first lid 120 can be permanently affixed to the frame 105, for example, hingedly coupled and movable between a sealed and an unsealed configuration. As discussed in further detail herein, the first lid 120 and/or the second lid 124 may be configured to house at least one valve, such as the valve 130 shown in FIGs. IA and IB. In some instances, the first lid 120 and/or the second lid 125 can be non-sealably coupled to the frame 105, for example, when the hyperbaric medical chamber 100 is in the collapsed configuration and/or for transport.

Valve 130 is configured to regulate the flow of fluids such as gases into or out of the hyperbaric medical chamber 100. By allowing the regulation of fluids, a pressure level inside the chamber 100 can be maintained, to prevent leaks or over-pressurization. By opening, closing, or modulating flow paths, the valve 130 ensures that the chamber operates within safe limits, enabling efficient performance of the system when in use (e.g., in the collapsed configuration). Depending on the design, the valve 130 may be configured for manual or automated response to pressure changes, acting as a relief mechanism to the hyperbaric medical chamber and maintaining operating conditions. The valve 130 is configured to release excess pressure when necessary to prevent damage or failure of the hyperbaric medical chamber 100 and associated equipment and/or conditions dangerous to the patient. For example, valve 130 can be or include a pop-off valve.

The hyperbaric medical chamber 100 is configured such that the frame 105 is coupled to the shell 110 and the rail 115. By coupling the frame 105 to the shell 110, when the frame expands in the lengthwise direction, L, the shell 110 is constrained to a maximum dimension of the frame 105 and/or vice versa. For example, in some embodiments, the shell 110 can include orbe coupled to an axial band (e.g., constructed of webbing cable, etc.) that defines a maximum length of the shell portion of the hyperbaric medical chamber 100 in the expanded configuration. Similarly, when the frame 105 collapses into a more portable size in the lengthwise direction, L, in diameter, D, or in both, the shell 110 remains constrained by the frame 105. The rail 115 is also constrained by the frame 105 in the lengthwise direction, such that the rail system 115 is the same length, L, at the frame no matter the length. This may be achieved by having a segmented rail, a telescopic rail, or other ways to shorten/lengthen the rail 115.

Acting functionally, the collapsed configuration of the hyperbaric medical chamber 100 enables portability. The combination of the lightweight components used, along with the collapsibility in the lengthwise direction and/or the diameter, allow for the transport and quick deployment of the hyperbaric medical chamber 100. This means that instead of having to transport a patient to the aforementioned treatment facilities, the treatment facility can be brought to the patient. Due to the lightweight and collapsible nature of the hyperbaric medical chamber 100, the frame 110, shell 115, and/orlid(s) 120, 125 may be carried by a single person, for example in a backpack configuration. In some instances, the lids 120, 125 can be transported/transportable separately from the frame 110 and shell 115 to reduce weight. Similarly, in some instances, gas sources backboards, and/or other 9 support components can be transported/transportable separately from frame 110 and shell 115.

Expanding and/or collapsing of the hyperbaric medical chamber can be performed manually, semi-automatically, or automatically. This means additional components may be used to motorize or facilitate transitions between the collapsed configuration and the expanded configuration. The hyperbaric medical chamber 100 enables quick deployment of a hyperbaric chamber, so great deference is given to techniques to accelerate the ease and speed at which the chamber may transition between configurations.

Acting functionally, the expanded configuration of the hyperbaric medical chamber 100 enables the encapsulation of a patient, along with thermoregulation equipment, pressurization or gas delivery equipment, and patient monitoring equipment.

The hyperbaric medical chamber 100 utilizes a combination of multiplace and monoplace mechanisms such that the shell 110 is pressurized with air or other gas, while a high oxygen concentration breathing gas is provided to the patient via a hood, mask or other suitable devices. Pressurizing the shell 110 with a gas containing about 21% or less oxygen (in some instances using only inert gases) can allow power electronics, heaters, and/or other devices to be contained within the envelope of the hyperbaric medical chamber 100 without posing substantial fire or bum risks to the patient as a high oxygen atmosphere would. Oxygen content within the shell 110 can be further managed through the use of a rebreather, as discussed below, which can reduce or eliminate exhaled oxygen from accumulating in the shell.

In contrast, known emergency and other monoplace hyperbaric chambers typically operate by pressurizing a volume of gas with a high oxygen concentration into which the patient is placed. In such known emergency and other monoplace hyperbaric chambers, substantial electronics and breathing components must pierce the envelope of the chamber to isolate such components from the high oxygen concentration because, for example, placing batteries and/or electronics within an oxygen-containing hyperbaric chamber produces an unacceptable risk of bums, fire, or explosion. Such fire safety measures substantially increase the bulk and reduce the portability of hyperbaric chambers, which can delay the availability of life-prolonging care. Known multiplace hyperbaric chambers are typically room-sized contraptions installed in fixed locations and/or are installed on ships or drilling platforms and generally make use of external air and/or oxygen sources, facility heating, hospital scale monitoring equipment, etc.

Embodiments described herein are highly portable. A gas cylinder that can be disposed within the hyperbaric medical chamber 100 can be operable to pressurize the hyperbaric medical chamber 100 with atmospheric gas or other gas with low or zero oxygen content. Breathing gas can be supplied by an oxygen supply, also disposed within the chamber via a SCUBA-style breathing circuit, in some instances including a rebreather. This use of low oxygen pressurization gas allows batteries, heaters, monitoring equipment, medication delivery devices, and other life monitoring and/or life prolonging equipment to be disposed within the envelope of the hyperbaric medical chamber 100, as discussed in further detail herein, without risk of fire or burn. This improves the transportability of the hyperbaric medical chamber 100, dramatically improving the ability to provide HBOT in emergency situations.

As discussed, the length, L, of the pod is expandable/collapsible property. In some embodiments, the diameter of the pod, D, is also expandable/collapsible. This can be accomplished through telescopic techniques, fragmentation of components, hinged components, origami-inspired folding, rolling or coiling techniques, detachable components, compression mechanisms, inflatable structures, interlocking elements, articulated linkages, or other suitable methods.

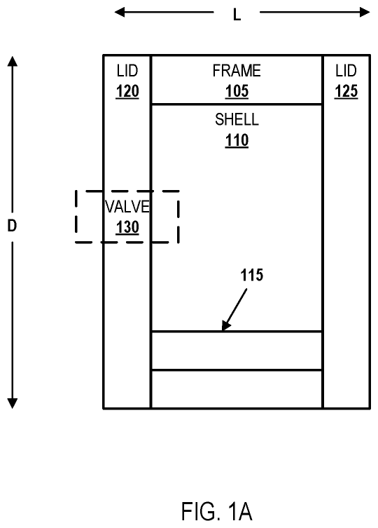

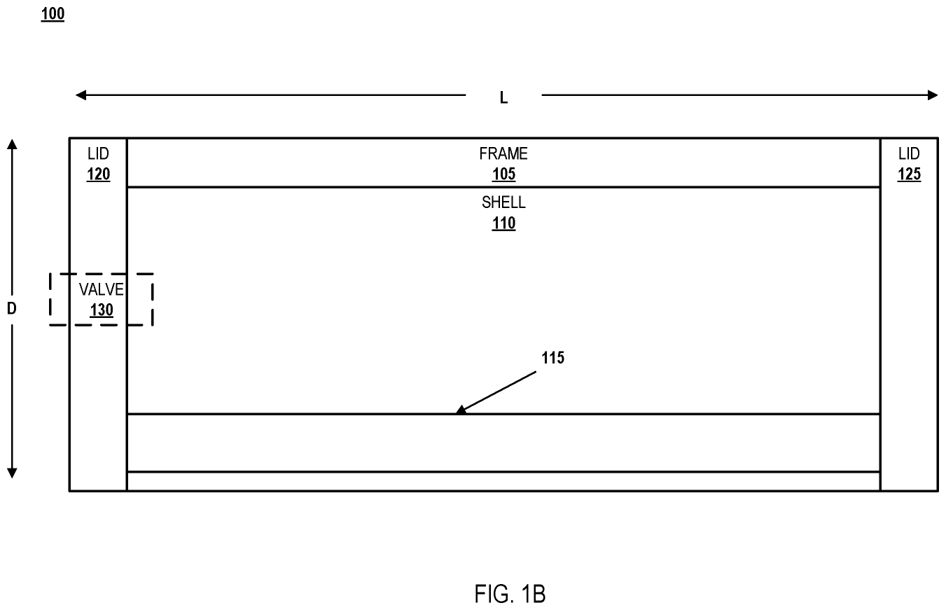

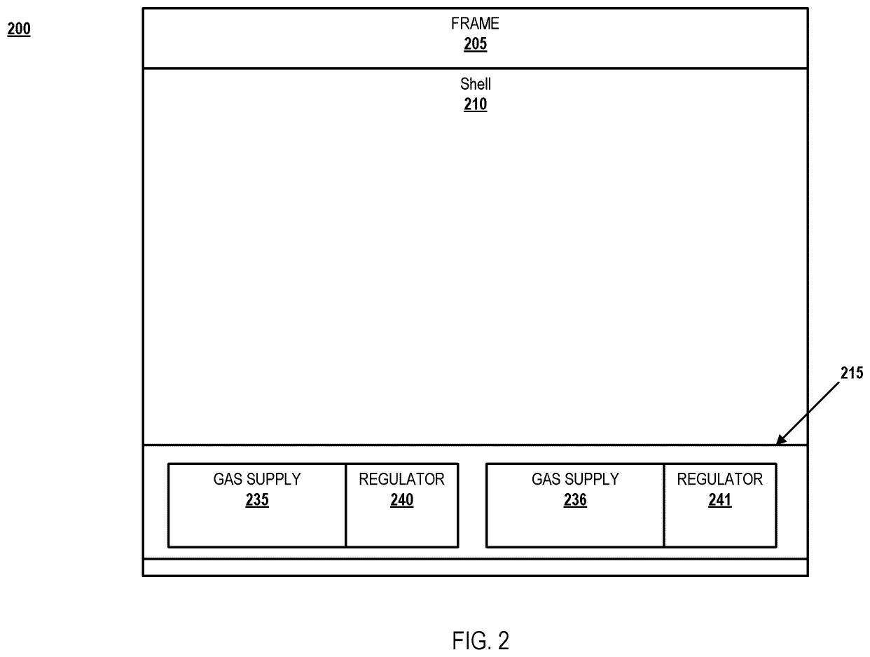

FIG. 2 is a block diagram of a portable hyperbaric medical chamber 200, according to an embodiment. The portable hyperbaric medical chamber 200 includes a frame 205, a shell 210, a rail 215, which can be structurally and/or functionally similar to similar features described above with reference to FIGS. IA and IB. Portable hyperbaric medical chamber 200 also includes a first gas supply 235, a second gas supply 236, a first regulator 240, and a second regulator 241.

In operation, the first gas supply 235 and second gas supply 236 (also referred to herein as tanks) act to store suitable gases, typically in a pressurized form. It can be advantageous to store pressurization gas (e.g., air) and breathing gas (e.g., oxygen) separately. Thus, the first gas supply 235 can contain pressurized air, nitrogen, or other suitable pressurization gas (e.g., a gas having less than 25% oxygen), and the second gas supply 236 can be operable to contain a high oxygen breathing gas (e.g., a gas containing greater than 21% oxygen, 32% oxygen, 36% oxygen, 50% oxygen, 95% oxygen, 99% or greater oxygen, or any other suitable oxygen concentration for hyperbaric treatment). As discussed in further detail herein, the first gas supply 235 is typically configured to deliver gas into the bulk volume of the portable hyperbaric medical chamber 200 and the second gas supply 236 is typically configured to deliver breathing gas directly to the patient. The first gas supply 235 and/or second gas supply 236 may be integrated with a monitoring system to detect leaks and maintain pressure levels to ensure reliable and safe pressurization and delivery of breathing gas to patients. Additionally, the first gas supply 235 and second gas supply 236 may be coupled to regulators to regulate the supply of gas. In embodiments in which air is the pressurization gas, the hyperbaric medical chamber 100 has fail-safe features, as the lowest possible oxygen concentration to which the patient can be exposed is about 21%. It may, in some instances, however, be advantageous to use a gas containing less or no oxygen to effectively eliminate the possibility of fire within the hyperbaric medical chamber 100 despite the flow of high concentration oxygen into the patient's breathing circuit, for example when an open breathing circuit, rather than a rebreather is employed. Patient safety can be provided in normal operation by the high oxygen breathing gas supplied via the second gas supply 236. Breathing gas concentration can be monitored by one or more sensors and, in the event of low oxygen, emergency venting of the inert gas and/or unsealing of the hyperbaric medical chamber 100 can be performed automatically and/or manually from within or exterior to the hyperbaric medical chamber 100.

In operation, the first regulator 240 and second regulator 241 act to control the flow and pressure of oxygen, air, or gas from the gas supplies 235, 236 to ensure the contents of the gas supplies 235, 236 are delivered safely and at an appropriate pressure and/or rate for the user and/or pressurization of the chamber 200. The gas supplies 235, 236 store oxygen, air, and/or gas at a high pressure, which the regulators 240,241 reduce to a safe and effective level. The regulators 240, 241 can be preset and/or adjustable. The first regulator 240 can be configured to pressurize the shell 210 to a target pressure with gas (e.g., air, nitrogen, etc.) from the first gas supply 235, and the second regulator 241 can be configured to deliver oxygen to the patient on demand and/or at a target flow rate when the shell is pressurized.

The rail 215 compartmentalizes the chamber 200 into a lower compartment and an upper compartment. The first gas supply 235 and the second gas supply 236 and/or regulators 240,241 can be stored in the lower compartment. Additionally, other components providing patient support can be stored in the lower compartment, which allows the patient to occupy the upper compartment without being impeded by or having an opportunity to interfere with gas supplies, regulators, and/or other support equipment. Alternatively, the compartments can be switched so the patient is in the lower compartment and the supportive equipment is in the upper compartment. Any suitable division and/or compartmentalization of the interior of the chamber 200 is recognized. Alternatively, compartmentalization may occur so that there is a compartment external to the shell 210 and within the frame 205, or external to the shell 210 and the frame 205 housing the support equipment, allowing for the patient to occupy the entirety of the chamber 200.

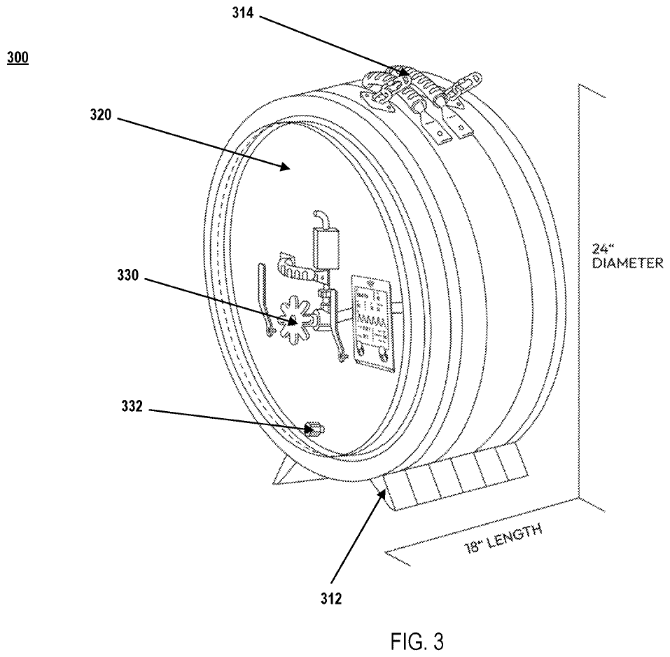

FIG. 3 is a perspective view of a hyperbaric medical chamber 300, according to an embodiment. In the illustration of FIG. 3, the hyperbaric medical chamber 300 is in a collapsed configuration. The chamber 300 of FIG. 3 may include components, such as lid 320 which is structurally and/or functionally similar to those of FIGs. IA, IB, and 2. As shown, in the collapsed configuration, the hyperbaric medical chamber 300 can have a diameter of approximately 24 inches and a length of approximately 18 inches. The length is limited by the thickness of stacking components (e.g., a first lid, a second lid, a collapsed length of the frame/rail/shell, or shell). The embodiment of FIG. 3 highlights a base or stand 312 and a handle 314. In some embodiments, the base 312 and/or handle 314 can be operable to secure the hyperbaric medical chamber 300 into the collapsed configuration. The base or stand 312 is a structure to stabilize the hyperbaric medical chamber 300. The base or stand 312 may be affixed to components of the chamber 300 or may be detachable. The base or stand 312 may be coupled to, or removably coupled to the lid 320, a frame of the chamber 300, or a second lid or base of the chamber 300. Alternatively, the hyperbaric medical chamber 300 may rest upon a completely separated base or stand 312.

The pressure relief valve 332 is a safety device that can be coupled to the lid 320 as shown in FIG. 3 or to the shell. The pressure relief valve 332 is configured to release excess pressure from the inside of the hyperbaric chamber to prevent damage or failure. For example, the pressure relief valve 332 ensures that the interior of the hyperbaric medical chamber 300, when in use, does not exceed safe operating limits. If the pressure inside the chamber rises beyond the predetermined threshold, the valve automatically opens, allowing air or another gas to escape. Once the pressure within the chamber 300 drops to a safe level, the valve closes and facilitates maintaining controlled conditions within the chamber 300. In addition or alternatively, the pressure release valve 332 can be operable to depressurize the shell after use.

In operation, the components of FIG. 3 are similar to those of FIG IA and allow for collapsibility of the components for transportation of the hyperbaric medical chamber 300. To facilitate the transportation of the chamber 300, the handle 314 enables ergonomic pickup of the hyperbaric medical chamber 300. The handle 314 may be singular or a plurality (shown) and may be affixed to any external portion of the hyperbaric medical chamber 300.

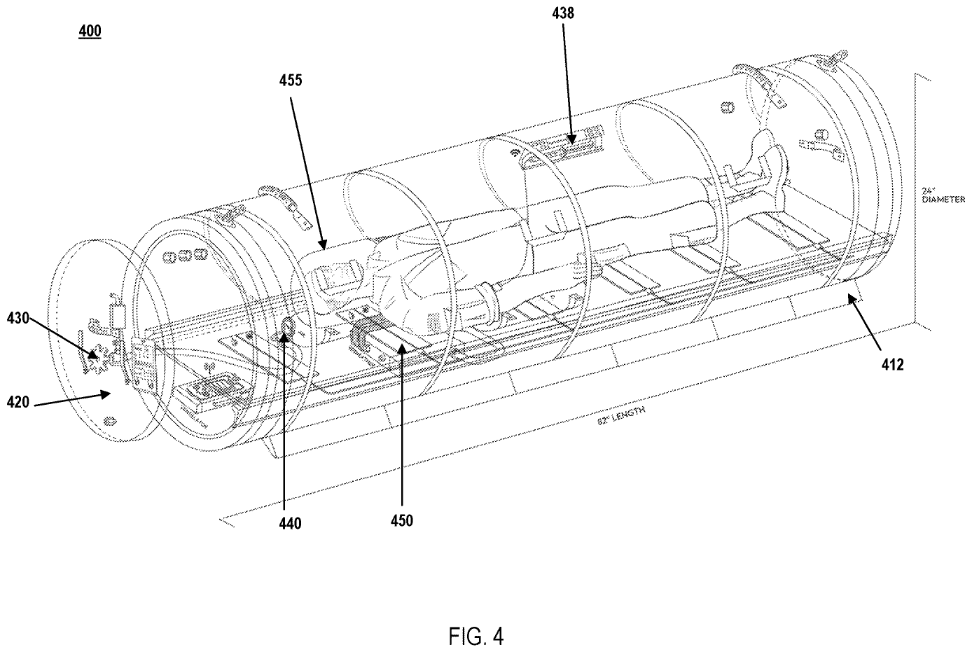

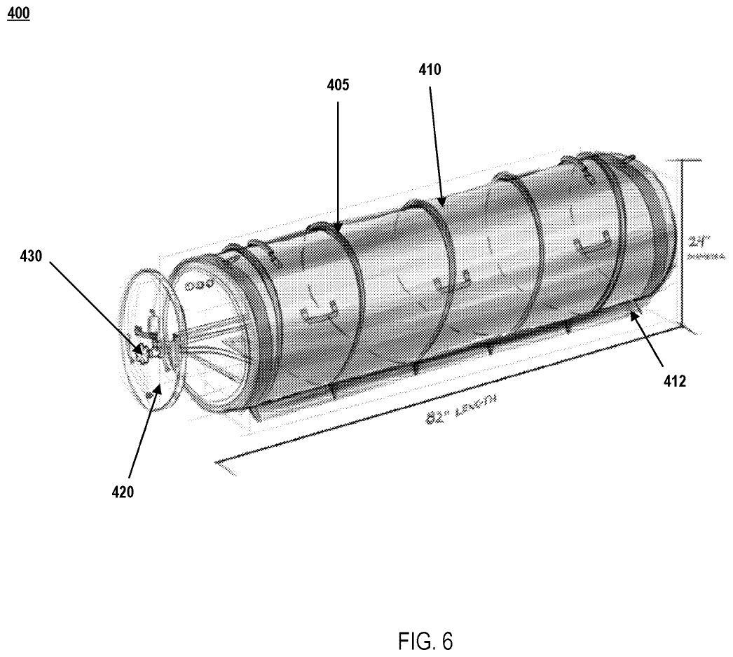

FIGS. 4, 5, and 6 depict an embodiment of a hyperbaric medical chamber 400 in a transparent perspective view, a perspective view, and a bottom view respectively. Hyperbaric medical chamber 400 includes a shell 410, a base 412, a lid 420, a valve 430, a first tank 435, a first regulator 440, and a second tank 436 that can be structurally and/or functionally similar to the corresponding features discussed above with reference to FIGS. 1, 2 and 3. FIG. 4 also depicts a rebreather 450, a patient hood 455, and a medication delivery device 438.

The first tank 435 may be an air tank, or other gas tank suitable for environmental control, particularly to pressurize shell 410. Two air tanks 435 are shown in FIGS. 4, 5 and 6, but the hyperbaric medical chamber 400 can contain any suitable number of air tanks 435, although it may be beneficial to have at least two air tanks 435 for redundancy and/or reserve.

The rebreather 450 is a type of regulator device that enables HBOT via the second (e.g., oxygen) tank 436, while the shell 410 is pressurized via the first (e.g., air) tank 435. As shown in FIGS. 4, 5, and 6, the rebreather 450 is coupled to the patient hood 455, but in alternative embodiments, can be coupled to a nasal cannula, face mask, and/or other suitable device to deliver oxygen to the breathing passages of the patient. Similar to with the air tank 435, it may be beneficial to have at least two second (e.g., oxygen) tanks 436 for redundancy and/or reserve.

The patient hood 455 is a transparent, lightweight, and airtight headgear configured to deliver breathing gas (typically high concentration and/or pure oxygen) to a patient. Because breathing 100% oxygen at high pressures (e.g., 2-3 atmospheres) enhances healing, the patient hood is configured to fit a patient and deliver 100% oxygen directly to the hood. In some embodiments, the hood 455 may form a seal on the neck of the patient, which can facilitate breath sensing and a demand-based supply of breathing gas from the rebreather 450. In other embodiments, the hood 455 may not seal to the patient, but may instead serve to create a pocket in which breathing gas collects from the oxygen tank 435.

The rebreather 450 can be operable to allow exhaled oxygen to be reused, which can decrease the needed size of the oxygen tank 436. As the patient inhales and exhales through the loop, carbon dioxide is removed using a scrubber, and oxygen levels are replenished to maintain a constant concentration. The advantage of using a rebreather in this manner is that extended oxygen delivery times are achieved and are more efficient than open-circuit air systems. In some embodiments, the rebreather can include a scrubber operable to remove carbon dioxide from the exhaled breath. Exhaled gases can be expelled from the rebreather 450 and/or the hood 455 into the pressurized shell 410. In some embodiments (e.g., in which the rebreather circuit does not contain a carbon dioxide scrubber) a scrubber can be located apart from the rebreather circuit and in the interior volume of the hyperbaric medical chamber 400.

Although FIGS. 4, 5 and 6 show and describe a rebreather, alternative embodiments can include an open-circuit demand regulator without rebreathing (e.g., with exhalant passing directly out of the hood 455 and into the pressurized shell), a constant flow regulator, or any other suitable breathing circuit operable to supply oxygen as needed to ensure that the patient breathes a safe and controlled gas mixture, typically a breathing mixture with high oxygen concentration, up to 100% oxygen for effective HBOT.

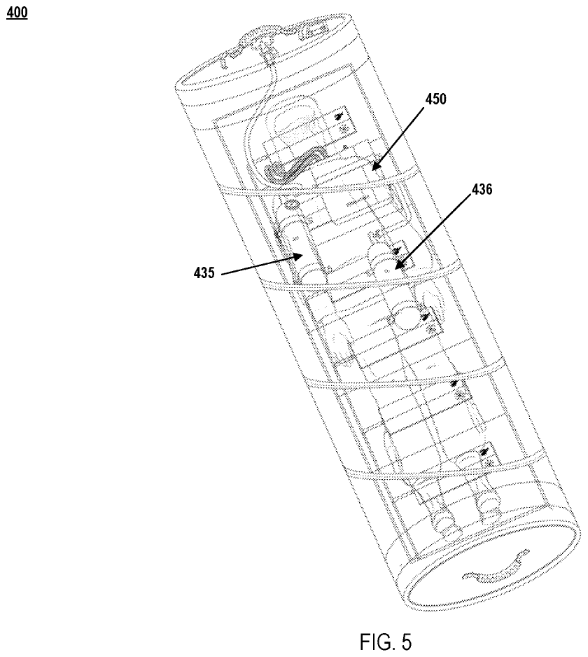

As shown best in FIG. 5, the air tank 435 and the oxygen tank 436 can be located in a bottom compartment, beneath the patient backboard. Such a location may ease patient loading, for example, so that bulky and heavy tanks and accompanying tubes and other devices are located out of the way and outside of the patient portion of the hyperbaric medical chamber 400.

Hyperbaric medical chamber 400 also includes a medication delivery device 438. Medication delivery device 438 can include crystalloid solutions and/or medications operable to be delivered to the patient, typically intravenously, as a bolus and/or infusion. The medication delivery device 438 can include or be coupled to patient monitors, such as blood pressure monitors, heart rate monitors, pulse oximeters, body temperature monitors, End-Tidal Capnographs, EKG leads, and/or any other suitable sensors. The medication delivery device 438 can be operable to deliver medications and/or fluids automatically, semi-automatically, and/or in response to a medical provider's direction in real time or substantially real time (e.g., within 5 minutes). For example, patient monitor(s) and/or the medication delivery device 438 can include a wireless transceiver operable to, for example, wirelessly transmit patient data to a receiver outside the hyperbaric medical chamber 400 continuously, in real time, and/or in a packetized manner. The medication delivery device 438 can be operable to receive commands to deliver medication from an external controller and cause appropriate medication that is stored onboard and within the shell 410 to be delivered in response to such commands, without requiring a medical provider to physically access the space containing the patient within the shell 410. In addition or alternatively, the medication delivery device can be operable to deliver medications in response to a patient's condition (e.g., blood pressure, heart rate, etc.). In some embodiments, medication delivery system 438 can include blood transfusion supplies and equipment and/or saline solution. In such embodiments, the medication delivery system 438 can be operable to monitor patient blood pressure and adjust patient blood pressure via transfusion of blood products, fluid, and/or vasoactive medications to maintain patient blood pressure within a target range.

Although not shown in FIGS. 4, 5 and 6, the hyperbaric medical chamber 400 can also include climate control sensors, heaters, and/or coolers. For example, the hyperbaric medical chamber 400 can include a power supply (e.g., a battery), electric heater, and thermoelectric cooler and be configured to maintain a predefined and/or configurable temperature best suited for a patient with a traumatic injury. Hypothermia in the trauma patient is detrimental and is a pillar of the lethal triad. Preventing hypothermia is paramount for increasing the chance of survivability for the patient. Shell 410 can be, include, or be surrounded by an insulator suitable to allow the heaters and/or coolers to maintain the interior of the hyperbaric medical chamber 400 at the human thermoneutral zone, including in below freezing conditions, in or under water, and/or in tropical conditions.

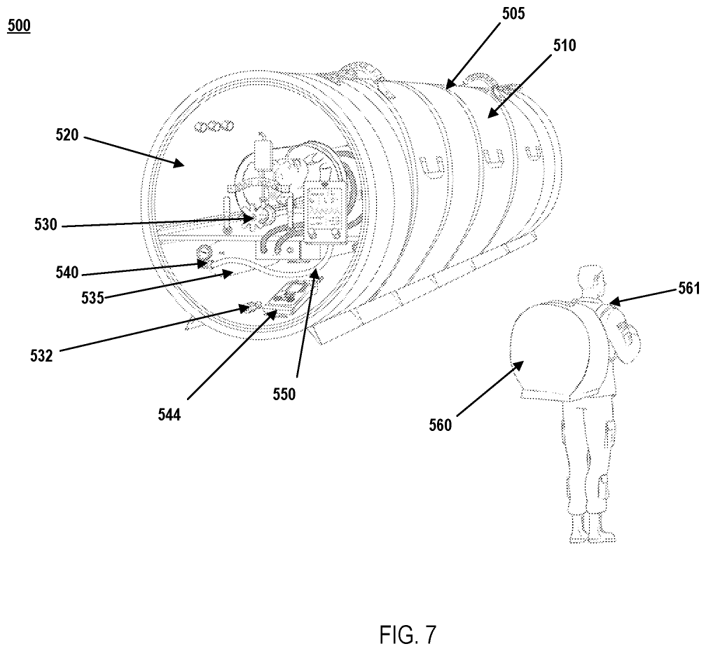

FIG. 7 depicts an embodiment of a hyperbaric medical chamber 500 in the context of both an expanded configuration and a collapsed configuration. The expanded configuration of FIG. 7 includes a frame 505, a shell 510, a lid 520, a valve 530, a pressure relief valve 532, an air tank 535, a regulator 540, a control unit 544, and a rebreather 550, each of which can be structurally and/or functionally similar to corresponding features discussed in detail above. The collapsed configuration of FIG. 7 shows a carrier 560 with a carrying strap 561.

The control unit 544 is a compact device that can be coupled to various systems associated with the hyperbaric medical chamber 500, such as a patient monitoring system, an air system, a climate control system, medication delivery system, an oxygen system and/or an actuation system. The control unit 544 includes a housing, a display, and an interface. A control unit 544 may provide wi-fi, Bluetooth, satellite, or other communications method connectivity. The control unit 544 FIG. 7 is enabled for wireless communication. In some embodiments, the control unit may provide feedback and/or control over oxygen levels, system status, and/or patient monitoring. Thus, in some instances, the hyperbaric medical chamber 500 can be fully operational without connections breaching the shell 510. Similarly stated, hyperbaric medical chamber 500 can be a fully self-contained unit in which most, wireless electronic signals enter the hyperbaric medical chamber 500 when in use.

The control unit 544 is couplable, through remote connectivity or through coupling such as hoses, cables, and connectors, to components used in conjunction with the hyperbaric medical chamber 500, such as the air tank 535, the regulator 540, and the rebreather 550. The control unit 544 may interface with an oxygen sensor located ambiently within the hyperbaric medical chamber pod 500 and within the breathing loop to measure gas concentration. The control unit 544 may be electrically coupled to solenoid valves to release gas from the airtank(s) 535. The control unit 544 may be coupled to pressure sensors to monitor the air tank(s) 535 and ambient pressure. The control unit 544 may be configured to display all pertinent information about the patient, the component status, and the environmental conditions within the medical chamber 500 and/or send status information to an external monitoring device.

In the example of FIG. 7, in operation, the control unit and/or rebreather 550 can periodically and/or continuously analyze gas concentrations and adjust oxygen levels by opening and/or closing solenoid valves to ensure the partial pressure of oxygen stays within a predetermined range based on preset parameters. The control unit 544 may also trigger an alarm if it detects a deviation in gas levels, low gas levels, low battery, equipment malfunction, hypoxia, hyperoxia, or other dangerous situations.

The carrier 560 can be a structure or cover for the hyperbaric medical chamber 500, or in the alternative, a configuration or reconfiguration of the hyperbaric medical chamber 500. In the instance of the carrier 560 being a structure or cover, the carrier 560 is configured to encapsulate the hyperbaric medical chamber 500. When the carrier 560 is a structure or cover, the structure or cover may be either a soft-shell or hard-shell carrying case that may provide protection from the elements, ergonomics for carrying the carrier with the hyperbaric medical chamber 500 inside, and protects the hyperbaric medical chamber 500 from damage. As shown, the shell 510, frame 505, lid 520, and carrier 560 can be man portable, weighing, for example, less than 70 pounds. In some embodiments, the hyperbaric medical chamber 500, including electronics and plumbing (but excluding gas containers) can weigh less than 70 pounds and fold into a backpack-sized collapsed configuration.

In other embodiments, the carrier 560 simply is a configuration of the hyperbaric medical chamber 500, whereas the hyperbaric medical chamber 500 is in its collapsed configuration. This may allow for the attachment of a carrying strap 561 or a carrying strap 561 may already be attached to the lid(s) 520 of the hyperbaric medical chamber 500. Alternatively, the carrier 560 may be a reconfiguration of the hyperbaric medical chamber 500, where the lid is inside-out to provide the functionality of element protection, ergonomics, and/or damage protection.

The carrying strap 561 is a soft structure that can be removably coupled to or fixedly coupled to the carrier 560, to the lid 520, or to the shell 510 in at least one place. The carrying strap 561 may be made of any durable material and is configured to withstand the loading of the weight of the hyperbaric medical chamber 500. Functionally, a user can thread their arms or other parts of their body through a gap created by the dual attachment of the strap to the carrier 560. This allows a user to wear the carrier and/or hyperbaric medical chamber 500 on their torso like a backpack, as shown in FIG. 7.

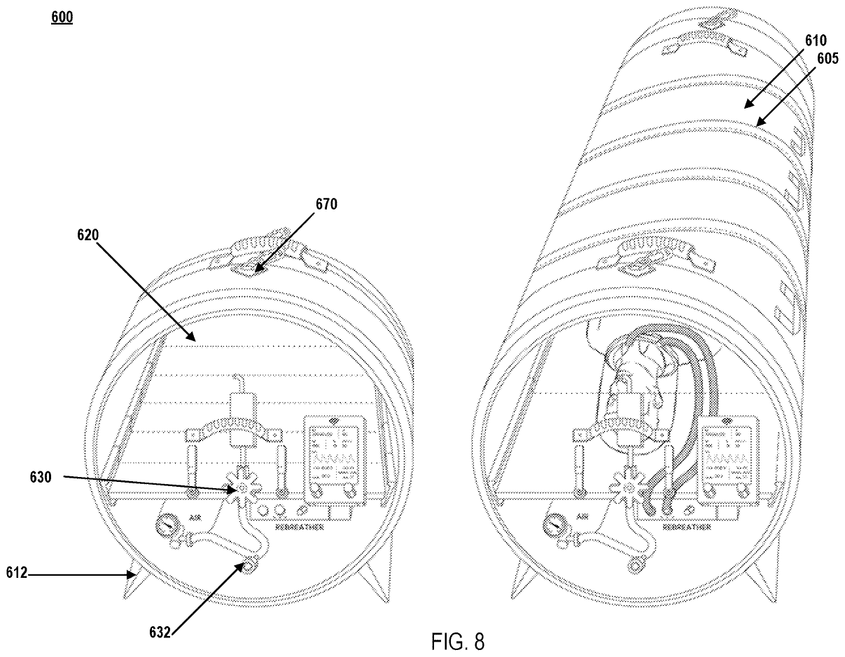

FIG. 8 illustrates a hyperbaric medical chamber 600 in both a collapsed configuration and an expanded configuration, according to an embodiment. The collapsed configuration includes a base 612, a lid 620, a valve 630, and a pressure relief valve 632. The expanded configuration of FIG. 8 includes a frame 605 and a shell 610. Each of these features can be structurally and/or functionally similar to the corresponding features described in detail above.

A hook(s) 670, shown in FIG. 8 is a fastening device configured to secure the hyperbaric medical chamber 600 in a collapsed configuration. Upon releasing the hook 670, the hyperbaric medical chamber 600 can be expanded or expanded automatically (e.g., at least partially via a spring force stored in the frame 610 urging the hyperbaric medical chamber 600 towards the expanded configuration. The hook(s) 670 may be fixed at either end of the frame 605, along the shell 610, or on the lid(s) 620. The hook may come in a variety of designs, including but not limited to, a bracket, a pipe u-bolt, a hook, ans hook, a snap hook, a carabiner, or other suitable fastening mechanism.

The hook(s) 670 of FIG. 8 allow for the hyperbaric medical chamber 600 to be evacuated rapidly. For example, the hook(s) 670 enable the attachment of cables from a helicopter to the hyperbaric medical chamber 600. This allows a patient inside the hyperbaric medical chamber 600 to be transported great distances at a fast pace without disturbing the ongoing HBOT. In other applications, the hook can be used to couple to a boat, a backpack, or an external lifting system or wench. The hook(s) 670 may be constructed of any material designed to withstand supporting the weight of the hyperbaric medical chamber 600 loaded with a patient.

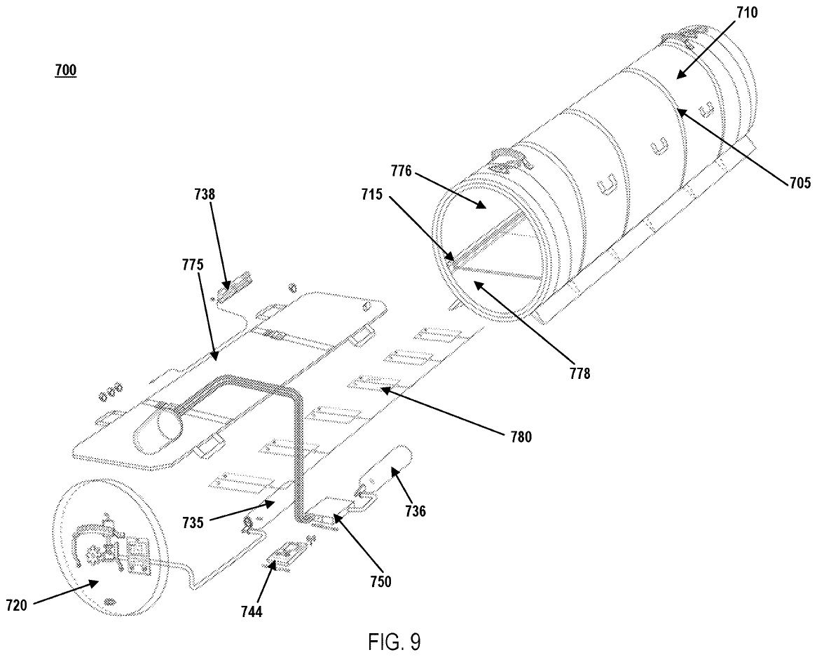

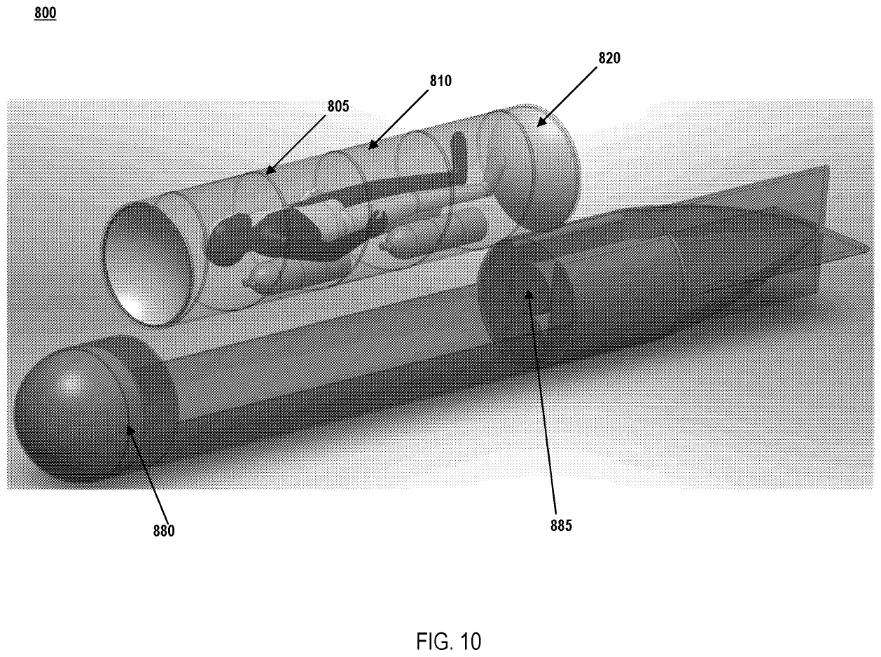

FIG. 9 is an exploded view of a hyperbaric medical chamber 700, according to an embodiment. The example hyperbaric medical chamber 700 includes a frame 705 a shell 710, a rail 715, an air tank 735, an oxygen tank 736, a medication delivery device 738, and a removably couplable lid 720, each of which can be structurally and/or functionally similar to corresponding features discussed in detail above. FIG. 9 highlights a backboard 775, which can compartmentalize the interior of the hyperbaric medical chamber 700 into a top compartment 776 and a bottom compartment 778.

The backboard 775 of FIG. 9 is configured to support a patient, for example, in an immobile position as a precautionary measure and/or in response to cervical or spinal injuries. For ease of supporting a patient, the backboard 775 may be compatible with straps for retaining and/or restraining a patient, and the backboard 775 may have handles to assist in carrying the backboard 775 with a patient on the backboard 775. The backboard is configured to be slidably disposed into the hyperbaric medical chamber 700 via rail 715. This can be done through a coupling mechanism or through the rail 715 being notched to accept the backboard 775. The rail 715 may use rollers, springs, or other components to make sliding of the backboard 775 into the rail 715 easier. The backboard 775 may be collapsible as well via any of the previous collapsing mechanisms discussed, such as folding, telescoping, interlocking, or other collapsible methods. In some embodiments, moving the shell 710 from the collapsed configuration to the expanded configuration can automatically cause the rail 715 to telescope and expand without the need for any further manual operations. In other embodiments, some or all of the rail 715 can be manually assembled after expanding the shell 710. For example, shelves or other supporting structures can be laid after air tank 735 and/or oxygen tank 736 are inserted.

Functionally, the rail 715 allows for the backboard 775 to slide entirely into the pod 700 so that the patient is inside the hyperbaric medical chamber 700. The lid 720 is removably couplable to the frame 705 of the pod 700 so as to hermetically seal with the pod 700 and encapsulate the patient within the hyperbaric chamber 700. The hermetic seal created between the frame 705 and the lid 720 can be accomplished in a number of ways/designs, including inserting the lid 720 into a ridge in the frame 705 and using the pressurization of the hyperbaric chamber 700 to seal the lid 720 to the frame 705 in an airtight fashion.

In operation, a patient may be loaded to the backboard 775. Optionally, the patient may be strapped and/or restrained on the backboard 775. In parallel, the collapsible hyperbaric medical chamber 700 may be deployed from a collapsed and portable configuration, such as that of FIGS. 7 and/or 8. Once in an expanded configuration, the pod 700 may be loaded with air tank(s), oxygen tank(s), regulators, rebreathers, patient monitoring equipment, communications equipment, etc. The equipment to be loaded into the hyperbaric medical chamber 700 occupies, for example, the lower compartment 778 of the hyperbaric medical chamber. The patient and backboard 775 are then inserted onto the rail 715 and slid along until the patient is securely confined within the hyperbaric medical chamber 700. The patient may be fitted with a hood as part of an oxygen delivery system, as discussed in conjunction with FIGS. 4, 5, and 6, and any oxygen valves can be opened. The air tank(s) are turned on to pressurize the hyperbaric medical chamber 700 (optionally via the control unit 744), and the lid 720 is aligned in place. Through the pressurization of the chamber 700, the lid 720 hermetically seals with the frame 705. Through the high-pressure air in the hyperbaric medical chamber 700 and the delivery of high concentration oxygen via the rebreather 750, hyperbaric oxygen therapy can be provided to the patient.

Thermoelectric heater/coolers 780 can be coupled to the rail 715 and positioned such that they span the length of the backboard 775. Thermoelectric heaters/coolers can (e.g., via control unit 744) maintain any suitable preset and/or configurable temperature of the patient and/or the atmosphere within the shell 710.For example, the heater/coolers 780 can contain or be communicatively coupled to sensors be operable to continuously monitor and adjusts the internal temperature, for example, in response to a patient's body temperature. In addition or alternatively, heater/coolers 780 can include a wireless transceiver, and/or or be communicatively coupled to control unit 744). For example, heater/coolers 780 and/or control unit 744 can include a wireless transceiver operable to, for example, wirelessly transmit patient data and/or internal temperature data to a receiver outside the hyperbaric medical chamber 700 continuously, in real time, and/or in a packetized manner. The heater/coolers 780 can be operable to receive commands to deliver increase or decrease temperature from an external controller in response to such commands, without requiring a medical provider to physically access the space containing the patient within the shell 710. In addition or alternatively, the heater/coolers 780 can be operable to increase or decrease temperature in response to a patient's condition (e.g., body temperature, blood pressure, heart rate, etc.).

FIG. 10 depicts an illustration of a hyperbaric medical chamber 800 configured to operate with a mobilizer, according to an embodiment. FIG. 10 depicts a hyperbaric chamber and mobilizer system 800. In the context of FIG. 10, the hyperbaric chamber is configured for use with a mobilizer. This could be used in a variety of scenarios where goals include stabilizing a patient and removing them from their environment because the hyperbaric chamber acts to stabilize the patient, which is insertable into the mobilizer. The mobilizer is a system to facilitate the movement of the hyperbaric chamber.

FIG. 10 includes a frame 805, shell 810, and lid(s) 820 of the hyperbaric chamber, disposed in a maritime mobilizer 880. The frame 805, shell 810, and lid(s) 820 of the hyperbaric chamber may have a similar form, fit, and function as that which is described in FIGS. 1-9, so as to allow for a quickly deployed collapsible hyperbaric chamber. The mobilizer 880 is configured to accept the expanded hyperbaric chamber through any of the following mechanisms: latches, notches, threading, snap-fit, friction locking, or other mating/attachment mechanisms. In some embodiments, the mobilizer can contain supplemental air and/or oxygen 885, which can be fluidically coupled to an interior of the shell 810 by any suitable means.

As shown in FIG. 10, a patient is disposed within the hyperbaric chamber and sufficiently stabilized, according to a method similar to that described with reference to FIG. 9. Once sufficiently stabilized, the hyperbaric chamber is loaded into the body 880 of the mobilizer system and attached through any of the above-listed mechanisms. Once the hyperbaric chamber is securely attached to the mobilizer 880, which can be used to transport the patient. The shell 810 can be pressurized to 2-4 atm (absolute). Thus, in some instances, the shell 810 can be operable to transport the patient at the surface and/or underwater at a depth of up to about 60 feet (corresponding to about 1.8 atmospheres of pressure) or to about 120 feet (corresponding to about 3.5 atmospheres of pressure) without additional reinforcement or structural materials to resist water pressure. Advantages of configuring the hyperbaric medical chamber to be used in conjunction with a mobilizer include speed and efficiency, access to remote areas, providing specialized medical service in transit and/or disaster/emergency response.

While various schematics, embodiments, and/or implementations have been described above, it should be understood that they have been presented by way of example only, and not limitation. Various modifications, changes, and/or variations in form and/or detail may be made without departing from the scope of the disclosure and/or without altering the function and/or advantages thereof unless expressly stated otherwise. For example, while embodiments described herein generally describe pressurized gas tanks, it should be understood that other suitable gas sources are possible. For example, a compressor could pressurize atmospheric air within a hyperbaric medical chamber. Likewise, while embodiments and/or features, components, configurations, aspects, etc. thereof may be described above in the context of certain implementations, it should be understood that such implementations are presented by way of example only and not limitation. Any of the embodiments and/or features, components, configurations, aspects, etc. thereof can be used in, and/or adapted for use in, other implementations unless expressly stated otherwise. Functionally equivalent embodiments, implementations, and/or methods, in addition to those described herein, will be apparent to those skilled in the art from the foregoing descriptions and are intended to fall within the scope of the disclosure.

Where schematics, embodiments, and/or implementations described above indicate certain components arranged in certain orientations, configurations, or positions, the arrangement of components may be modified. Although various embodiments have been described as having particular features, configurations, and/or combinations of components, other embodiments are possible having a combination of any features, configurations, and/or components from any of the embodiments described herein, except mutually exclusive combinations. The embodiments described herein can include various combinations and/or sub-combinations of the functions, components, configurations, and/or features of the different embodiments described.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods described above indicate certain events occurring in certain order, the ordering of certain events may be modified. Additionally, certain events may be performed concurrently in a parallel process, when possible, as well as performed sequentially as described above. While methods have been described as having particular steps and/or combinations of steps, other methods are possible having a combination of any steps from any of the methods described herein, except mutually exclusive combinations and/or unless the context clearly states otherwise.

What is claimed is:

1. An apparatus, comprising:
a frame, the frame configured to transition between a collapsed configuration to an expanded configuration;
a collapsible shell coupled to the frame, the collapsible shell defining an internal compartment when the frame is in the expanded configuration, the internal compartment configured to be pressurized;
a collapsible rail disposed inside the collapsible shell and coupled to the frame;
a lid removably coupled to the collapsible shell at a first end to hermetically seal the collapsible shell when the frame is in the expanded configuration.

2. The apparatus of claim 1, further comprising a valve coupled to the lid and configured to regulate a pressure within the internal compartment.

3. The apparatus of claim 1, further comprising a gas supply configured to pressurize the internal compartment.

4. The apparatus of claim 1, further comprising:
a first gas supply configured to pressurize the internal compartment; and
a second gas supply configured to provide a breathing gas to a patient within the internal compartment when the internal compartment is pressurized by the first gas supply.

5. The apparatus of claim 4, wherein the first gas supply contains compressed air and the second gas supply contains at least 95% oxygen.

6. The apparatus of claim 1, wherein the collapsible rail is collapsible through a telescoping mechanism.

7. The apparatus of claim 1, wherein the collapsible rail is configured to support a patient.

8. The apparatus of claim 1, wherein the frame has a form of a spiral wound around the shell and is configured to exert a spring force tending to urge the frame into the expanded configuration when the frame is in the collapsed configuration.

9. The apparatus of claim 1, wherein pressurizing the internal compartment exerts a force on the lid to cause the lid to hermetically seal to the collapsible shell.

10. A system, comprising:
a hyperbaric chamber including:
a collapsible frame configured to transition between an expanded configuration and a collapsed configuration;
a flexible shell attached to the collapsible frame to define an inside of the hyperbaric chamber, the flexible shell and the collapsable frame collectively configured to contain a pressurized internal environmental control; and
a rail disposed inside the flexible shell and coupled to the collapsible frame, a top portion of the hyperbaric chamber being above the rail and a bottom portion of the hyperbaric chamber being below the rail;
a gas supply configured to pressurize the inside of the hyperbaric chamber with at least one of oxygen or air, the gas supply disposed in the bottom portion of the hyperbaric chamber; and
a regulator coupled to the gas supply to control the pressure inside the hyperbaric chamber.

11. The system of 10, wherein the flexible shell is constructed from a high-performance multifilament yarn spun from liquid crystal polymer.

12. The system of 10, wherein the gas supply is configured to pressurize the inside of the hyperbaric chamber with air.

13. The system of claim 10, wherein the gas supply is a first gas supply configured to pressurize the inside of the hyperbaric chamber with a gas that contains less than 25% oxygen, the system further comprising:
a second gas supply disposed in the bottom portion of the hyperbaric chamber and configured to supply a gas that is at least 50% oxygen to a patient disposed in a top portion of the hyperbaric chamber.

14. The system of claim 13, wherein the second gas supply is configured to supply the gas that is at least 50% oxygen to the patient via at least one of a cannula, a mask, or a hood.

15. The system of claim 10, further comprising a backboard resting on the rail when the collapsible frame is in the expanded configuration, the gas supply being disposed below the backboard, the backboard configured to support a patient in the top portion of the hyperbaric chamber.

16. A system, comprising:
a hyperbaric chamber configured to be pressurized to at least 2 atmospheres, the hyperbaric chamber including:
a collapsible frame configured to transition between an expanded configuration and a collapsed configuration;
a flexible shell attached the collapsible frame to define an inside of the hyperbaric chamber, the flexible shell and the collapsable frame collectively configured to contain a pressurized internal environment; and
an oxygen source configured to supply a gas containing at least 95% oxygen to a pressure when the hyperbaric chamber is pressurized, the oxygen source disposed in the bottom portion of the hyperbaric chamber; and
at least one of a cannula, a mask, or a hood configured to cover an airway of a patient and coupled to the oxygen source.

17. The system of claim 16, further comprising a carbon dioxide scrubber configured to remove carbon dioxide from air exhaled by the patient.

18. The system of claim 16, further comprising a source of pressurization gas, the source of pressurization gas configured to pressurize the hyperbaric chamber to at least 2 atmospheres.

19. The system of claim 18, wherein the source of pressurization gas is disposed in the bottom portion of the hyperbaric chamber.

20. The system of 16, further comprising a rebreather, the rebreather configured to recycle exhaled oxygen and confine oxygen to a breathing circuit, and impede oxygen from being exhaled into a free volume of the pressurized internal environment of the hyperbaric chamber.
